(19) United States
(12) Patent Application Publication
(10) Pub. No.: US 2026/0126448 A1
(43) Pub. Date: May 7, 2026

(54) KIT FOR DETECTING SOLUBLE GROWTH STIMULATION EXPRESSED GENE 2 PROTEIN

(71) Applicant: Beijing Strong Biotechnologies, Inc., Beijing (CN)

(72) Inventors: Yao Liu, Beijing (CN); Xi Liu, Beijing (CN)

(21) Appl. No.: 19/428,030

(22) Filed: Dec. 19, 2025

Related U.S. Application Data
(62) Division of application No. 17/440,930, now Pat. No. 12,529,703.

(30) Foreign Application Priority Data
Mar. 28, 2019 (CN) 201910241518.7

(51) Int. Cl. G01N 33/68; G01N 21/31; G01N 33/543; G01N 35/00

(52) U.S. Cl. CPC G01N 33/68; G01N 21/31; G01N 33/54313; G01N 35/00

(57) ABSTRACT

The present disclosure relates to a kit for detecting a soluble growth stimulation expressed gene 2 protein. In particular, the present disclosure relates to a latex-enhanced turbidimetric immunoassay kit for detecting the concentration and/or content of the sST2 in human samples. The kit can be used in transmission immunoturbidimetry and scattering immunoturbidimetry. The kit comprises a buffer system, an anti-interference component, latex microspheres, an anti-sST2 antibody, etc. The latex-enhanced immunoturbidimetric agent of the present disclosure can detect sST2 proteins within a range of <400 ng/ml in a sample, with a sensitivity of up to 0.1 ng/ml and a high specificity, accuracy and precision. The kit is suitable for a fully automatic biochemical analyzer and a scattering analyzer, and has the advantages of convenient and fast use and low cost, and can be used clinically to detect the sST2 protein.

### CROSS-REFERENCE TO RELATED APPLICATIONS

This application is a divisional application of, and claims priority to, U.S. patent application Ser. No. 17/440,930, which was filed on Sep. 20, 2021, which is a U.S. National Phase Entry of International PCT Application No. PCT/CN2020/073461, which was filed on Jan. 21, 2020, and which claims the benefit of Chinese Patent Application Serial No. 201910241518.7, which was filed on Mar. 28, 2019. The contents of each of those applications are incorporated herein by reference in their entireties.

### TECHNICAL FIELD OF THE INVENTION

The present disclosure relates to a kit for detecting a soluble growth stimulation expressed gene2 protein and protein product (sST2) thereof inhuman blood. In particular; the present disclosure relates to a latex-enhanced turbidimetric immunoassay kit for detecting the concentration or content of the sST2. The kit can be used in transmission immunoturbidimetry and scattering immunoturbidimetry.

### BACKGROUND OF THE INVENTION

The isolation of murine specific growth stimulation expressed genes from BALB/c-3T3 cells was described by Tominaga; they called one of these genes “St2” (for growth stimulation expressed gene 2). The St2 gene encodes two protein products: ST2, which is a soluble secreted form; and ST2L, a form of transmembrane receptor very similar to the interleukin-1 receptor. The HUGO Nomenclature Committee refers to the human homologue as interleukin 1 receptor-like 1 (IL1R-L1).

ST2 is a member of the interleukin-1 receptor family and has two existing forms: transmembrane (ST2L) and soluble (sST2). Th (helper T cell) can secrete a variety of cytokines. Th cells express CD4, and the so-called CD4+ T cell refers to Th. HIV can specifically damage Th cells, causing destroyed immune system of patients. ST2L has immunomodulatory functions and plays an important role in T cell-mediated immune diseases such as asthma and rheumatoid arthritis.

ST2L neutralizing antibody or sST2 will block the binding of ST2L to ligands, thereby down-regulating Th2 (Th2 cells mainly secrete IL-4, IL-5, IL-6 and IL-10, etc., the main function thereof is to stimulate B cell proliferation and produce antibodies of immunoglobulin G1 and immunoglobulin E, and are related to humoral immunity). Lymphocyte function suggests that sST2 has an inhibitory effect on inflammation.

### SUMMARY OF THE INVENTION

According to some embodiments of the application, provided is a detection kit for soluble growth stimulation expressed gene 2 protein, which comprises:
a first reagent,
a second reagent,
optionally, calibrator(s),
optionally, control material(s).

In some embodiments, the first reagent comprises:
10 mmol/L to 500 mmol/L buffer, with pH range of 5.0 to 8.0,
10 mmol/L to 700 mmol/L dispersing agent,
0.05% to 5% w/v coagulant,
anti-interference composition, and
optionally, 0.05% to 0.5% w/v preservative.

In some embodiments, the second reagent comprises:
10 mmol/L to 500 mmol/L buffer,
0.05% to 0.25% w/v latex microspheres, and
anti-sST2 antibody or antigen-binding fragment thereof.

In some embodiments, the buffer in the first reagent and the second reagent is the one or combination thereof selected from the group consisting of: HEPES buffer, glycine buffer, Tris buffer, PBS buffer, MOPS buffer and boric acid buffer.

In specific embodiments, the buffer of the second reagent is boric acid buffer, pH 9.0.

In some embodiments, the dispersing agent is the one or combination thereof selected from the group consisting of: salt ion, thiocyanate, organic dispersant and surfactant.

In some embodiments, the coagulant is the one or combination thereof selected from the group consisting of: polyethylene glycol, methacryloyloxy phosphatidylcholine and polyether diamine.

In some embodiments, the preservative is the one or combination thereof selected from the group consisting of: azide and PC preservative.

In some embodiments, the surface functional group of the latex microspheres is selected from one of the following: carboxyl, sulfhydryl, chloromethyl or without surface functional group.

In some embodiments, the average particle size of the latex microspheres is 300 nm to 600 nm, preferably 350 nm to 450 nm.

In some embodiments, the anti-sST2 antibody is the one or combination thereof selected from the group consisting of: non-affinity IgG antibody, affinity IgG antibody and IgY antibody.

In some embodiments, the anti-sST2 antibody is derived from murine, rabbit, goat or avian.

In some embodiments, the anti-sST2 antibody is a monoclonal antibody or a polyclonal antibody.

In specific embodiments, the antigen-binding fragment is selected from: Fab, Fab′, (Fab′)2, Fv or scFv.

In some embodiments, the anti-sST2 antibody or antigen-binding fragment thereof is covalently bound to the surface of the latex microspheres.

In some embodiments, the anti-interference composition comprises: 0.1% to 10% w/v surfactant, and 0.5 KU/L to 10 KU/L lipid digesting enzyme.

In some embodiments, the surfactant is the one or combination thereof selected from the group consisting of Triton X, Tween, AEO, Thesit, Brij and NP.

In some embodiments, the lipid digestive enzyme is the one or combination thereof selected from the group consisting of lipase and triglyceride oxidase.

In some embodiments, the blocking agent on the surface of the latex microspheres is the one or combination thereof selected from the group consisting of: polyethylene glycol, polyethylene glycol polyamine, bovine serum albumin, dextran gel and casein.

In some embodiments, the first reagent also comprises a blocker; the blocker is the one or combination thereof selected from the group consisting of: murine serum, murine IgG, murine IgM, goat IgG and goat IgM; the concentration of the blocker is 0.10% to 5% w/v.

In some embodiments, the calibrator(s) comprise(s) sST2 of known concentration.

In specific embodiments, the calibrator(s) comprise(s) 25 ng/mL, 50 ng/mL, 100 ng/mL, 200 ng/mL or 400 ng/mL of sST2 protein.

In some embodiments, the control material(s) comprise(s) sST2 of known concentration. In specific embodiments, the control material(s) comprise(s): 30 ng/nL or 100 ng/mL of sST2 protein.

In some embodiments, the calibrator(s) or the control material(s) comprise(s) a buffer selected from the following: phosphate buffer, HEPES buffer, MOPS buffer, MES buffer and PIPES buffer. In some embodiments, the concentration of the buffer is 5 mmol/L to 250 mmol/L; pH is 4.0 to 8.0. The calibrator(s) or the control material(s) also comprise(s) 1.0% to 10% w/v protectant (for example selected from: bovine serum albumin, saccharide and alcohol). The calibrator(s) or the control material(s) also comprise(s) 0.05% to 1.5% ow/v preservative (for example selected from: sodium azide, PC and dithiothreitol).

In specific embodiments, provided is a detection kit for soluble growth stimulation expressed gene 2 protein, comprising:
the first reagent, comprising 200 mmol/L sodium chloride, 1% surfactant, 0.1% sodium azide and 100 mmol/l HPEPS buffer, pH 8.0;
the second reagent, comprising 0.1% w/v 400 nm latex microspheres and 30 μg/ml anti-sST2 monoclonal antibody.

In another specific embodiment, provided is a detection kit for soluble growth stimulation expressed gene 2 protein, comprising:
the first reagent, comprising 150 mmol/L sodium chloride, 1% surfactant, 0.1% sodium azide, 5 KU/L lipase, 0.5% methacryloyloxy phosphatidylcholineand 100 mmol/A Tris buffer, pH 7.4;
the second reagent, comprising 0.15% w/v 400 nm latex microspheres, 20 μg/ml sST2 antibody Fab fragment, 100 mmol/L glycine buffer pH 8.0, 0.1% NaCl, 0.5% methacryloyloxy phosphatidylcholine and 0.10% NaN₃.

In another specific embodiment, provided is a detection kit for soluble growth stimulation expressed gene 2 protein, comprising:
the first reagent, comprising 1% surfactant, 0.1% sodium azide, 5 KU/L lipase, 2% sodium cholate, 5 mmol/l CaCl₂), 2.0% methacryloyloxy phosphatidylcholine, 3% blocker HBR-8TM and 200 mmol/l Tris buffer, pH 7.4;
the second reagent, comprising 0.15% w/v 400 nm latex microspheres, 20 μg/ml sST2 antibody Fab fragment, 100 mmol/L glycine buffer pH 8.0, 0.1% NaCl, 0.5% methacryloyloxy phosphatidylcholine and 0.10% NaN₃.

The above kits optionally comprise calibrator(s) and/or control material(s), which comprise(s) sST2, 100 mmol/L phosphate buffer pH 5.5, 150 mmol/LNaCl, 5% BSA, 10% sorbitol and 0.1% NaN₃.

According to the embodiments of the present application, there is also provided an anti-interference composition, which comprises 0.1% to 10% w/v surfactant and 0.5 KU/L to 10 KU/L lipid digesting enzyme. The surfactant is the one or combination thereof selected from the group consisting of: Triton X, Tween, AEO, Thesit, Brij and NP. The lipid digestive enzyme is the one or combination thereof selected from the group consisting of lipase and triglyceride oxidase.

According to the embodiments of the present application, there is also provided use of the anti-interference composition in the preparation of an immuno-turbidimetric kit.

According to the embodiments of the present application, there is also provided a reagent, which comprises the anti-interference composition of the present application.

According to the embodiments of the present application, there is also provided an improved method for fixing polypeptides to latex microspheres, comprising the steps:
1) providing polypeptide, preferably providing polypeptide in a buffer;
2) providing latex microspheres, preferably providing latex microspheres in a buffer;
3) mixing the polypeptide with the latex microspheres at 10° C. to 35° C. (preferably 20° C. to 25° C.) (preferably by shaking and mixing) for 6 to 10 hours (preferably 7 to 9 hours) to obtain a first mixture;
4) providing a crosslinking agent, preferably carbodiimide;
5) contacting the crosslinking agent with the first mixture at 30° C. to 60° C. (preferably 45° C. to 55° C.) for 2 to 5 hours (preferably 2 to 4 hours) to obtain latex microspheres cross-linked with polypeptide.

In the context of the present application, step 1) and step 2) can be interchanged in order.

In the context of the present application, the step 4) can be interchanged with step 1), 2) and 3) in order, respectively.

In some embodiments, the polypeptide is a polypeptide carrying amino, carboxyl, hydroxyl or sulfhydryl; preferably, the polypeptide is selected from: antibody, antigen-binding fragment, antigen, enzyme and recombinant protein.

In some embodiments, the surface functional group of the latex microspheres is selected from one of the following: carboxyl, sulfhydryl, chloromethyl or without surface functional group.

In some embodiments, the average particle size of the latex microspheres is 300 nm to 600 nm, preferably 350 nm to 450 nm.

In some embodiments, the buffer in step 1) and step 2) is the one or combination thereof selected from the group consisting of: HEPES buffer, glycine buffer, Tris buffer, PBS buffer, MOPS buffer and boric acid buffer; the buffer provides pH of 7.0 to 9.0.

In specific embodiments, the buffer in step 1) and step 2) is boric acid buffer, pH9.0; the concentration of the buffer is 10 mmol/l to 1000 mmol/l.

In some embodiments, a blocking step is also included: contacting the latex microspheres cross-linked with polypeptide with a blocking system at 15° C. to 28° C. for 1 hour to 24 hours; in some embodiments, the blocking system comprises a blocking agent, a buffer with pH 5.0 to 8.0 and a surfactant. The blocking agent is the one or combination thereof selected from the group consisting of: 0.1% to 5.0% w/v polyethylene glycol with a molecular weight of 1000 to 5000; 0.1% to 5.0% w/v polyethylene glycol polyamine with a molecular weight of 1000 to 5000; 2.0% to 10.0% w/v bovine serum albumin; 2.0% to 10.0% w/v dextran gel; and 2.0% to 10.0% w/v casein. In some embodiments, the buffer is selected from: phosphate buffer, gly cine buffer and HEPES buffer. In some embodiments, the surfactant is the one or combination thereof selected from the group consisting of: 2.0% to 8.0% w/v Triton X, 2.0% to 8.0% Tween and 2.0% to 8.0% w/v AEO.

In some embodiments, after the blocking step, an optional step is further included, wherein the step is the one or combination thereof selected from the group consisting of rinsing the latex microspheres cross-linked with polypeptide, centrifuging and collecting the latex microspheres cross-linked with polypeptide, and packaging the latex microspheres cross-linked with polypeptide.

### DESCRIPTION OF THE FIGURES

FIG. 1: the effects of different cross-linking buffer systems on the absorbance of reagents after cross-linking.

FIG. 2: the effects of different cross-linking temperatures on the absorbance after cross-linking.

FIG. 3: the effects of different EDC activation temperatures on the absorbance of reagents.

FIG. 4: the effects of different antibodies on the absorbance of reagents after cross-linking.

FIG. 5: the effects of different blocking systems on absorbance of blank and sensitivity of the reagents.

FIG. 6: linearity results of the kit of the present disclosure.

FIG. 7: pre-zone results of the kit of the present disclosure.

FIG. 8: sensitivity results of the kit of the present disclosure.

FIG. 9: stability results of the kit of the present disclosure.

### DETAILED DESCRIPTION OF THE EMBODIMENTS

### Examples

### Example 1. The Effects of Buffer and pH on the Absorbance of Reagents

1. The first reagent:
   200 mmol/L sodium chloride,
   1% surfactant (Triton X 100),
   0.10% sodium azide,
   100 mmol/l HPEPS buffer, pH 8.0,
   the first reagent was a colorless or slightly yellow transparent liquid.
2. The second reagent:
   methods of preparation:
   (1) each of 0.5 mL latex microspheres with a particle size of 400 nm was added to 4.5 mL of boric acid pH 9.0, carbonic acid pH 9.0, HEPES pH 8.0, Tris pH 8.0, PBS pH 7.0 or HEPES pH 7.0 buffers, respectively, to obtain latex microsphere suspensions;
   (2) each of 1 mg anti-sST2 monoclonal antibody was added to 4.5 mL of boric acid pH 9.0, carbonic acid pH 9.0, HEPES pH 8.0, Tris pH 8.0, PBS pH 7.0 or HEPES pH 7.0 buffers, respectively, to obtain antibody solutions;
   (3) the antibody solutions and the latex microsphere suspensions were mixed at 20° C. by mixing and shaking for 8 hours;
   (4) 25 mg carbodiimide was dissolved in 8 mL deionized water to prepare and obtain a carbodiimide aqueous solution for later use;
   (5) the carbodiimide aqueous solution was added to the mixed solution of step (3) for reaction and activation at 60° C. for 4 hours to obtain cross-linked latex-antibody suspensions;
   (6) a blocking solution containing BSA was added for blocking overnight at room temperature (15° C. to 28° C.);
   (7) the mixed solutions were centrifuged at 4° C., 14000 rpm for 30 minutes;
   (8) the supernatant was removed and 50 ml working solution was added (100 mmol/L HEPES buffer pH 8.0, 0.1% NaCl, 1% sucrose and 0.1% NaN₃);
   (9) resuspended for later use.
3. Calibrator(s) and control material(s):
   sST2 recombinant antigen,
   100 mmol/L HEPES buffer pH 7.5,
   100 mmol/L NaCl,
   0.1% NaN₃.
4. Detection:
   detection tool: Beckman AU5800 automatic biochemical analyzer;
   analysis mode: FIXED;
   wave length: 660 nm;
   amount of samples: 10 μL; R1: 150 μL; R2: 50 μL;
   calibration method: SPLINE, 6-point calibration; reaction direction: ascending.

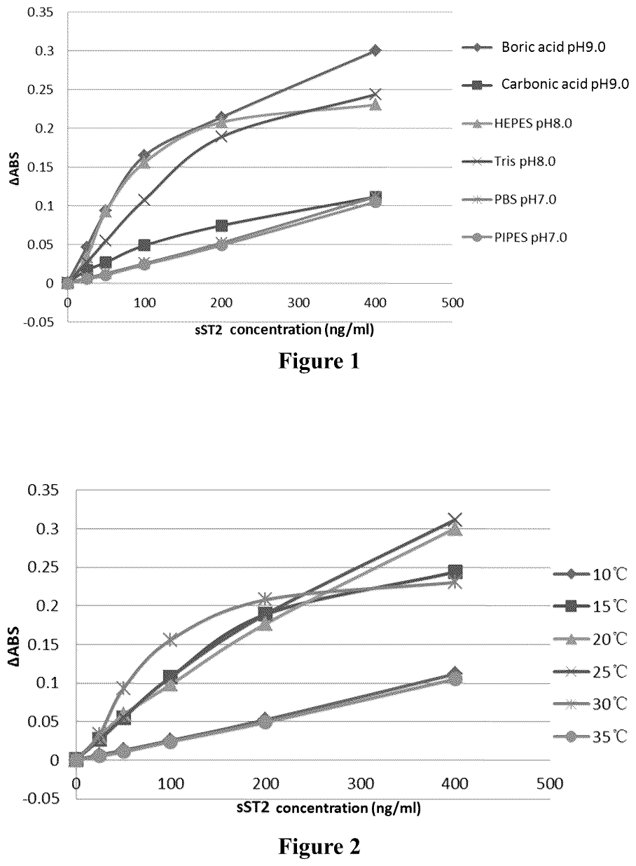

According to FIG. 1, it was shown that different buffer types and pH conditions had different effects on the absorbance of reagents. According to the results, preferably, boric acid buffer, pH 9.0 was the optimal cross-linking environment.

### Example 2. The Effects of Different Temperatures on the Absorbance of Reagents

1. The first reagent: same with Example 1.
2. The second reagent:
   (1) each of 0.5 mL latex microspheres with a particle size of 400 nm was added to 4.5 mL boric acid pH 9.0 buffer, respectively, to obtain latex microsphere suspensions;
   (2) each of 1 mg anti-sST2 monoclonal antibody was added to 4.5 mL boric acid pH 9.0 buffer, respectively, to obtain antibody solutions;
   (3) each of 2 ml of the antibody solutions and the latex microsphere suspensions were mixed at 10° C., 15° C., 20° C., 25° C., 30° C. and 35° C., respectively, by mixing and shaking for 8 hours;
   (4) to (7) were the same with Example 1;
   (8) the supernatant was removed, and each of 10 ml working solution was added (100 mmol/L HEPES buffer pH 8.0, 0.1% NaCl, 1% sucrose and 0.1% NaN₃);
   (9) resuspended for later use.
3. Calibrator(s) and control material(s):
   same with Example 1.
4. Detection: According to FIG. 2, it was shown that different temperatures had different effects on the absorbance of reagents. According to the results, preferably, boric acid buffer, 20° C. to 25° C. was the optimal cross-linking environment.

### Example 3. The Activation Effects of Different Temperatures on the Functional Groups of Latex

1. The first reagent: same with Example 1.
2. The second reagent:
   methods of preparation:
   (1) same with Example 2;
   (2) same with Example 2;
   (3) the antibody solutions and the latex microsphere suspensions were mixed and shaken at 25° C. for 8 hours;
   (4) same with Example 1;
   (5) the carbodiimide aqueous solution was added to the mixed solution of step (3), and each of 2 ml mixed solutions was placed at 30° C., 40° C., 45° C., 50° C., 55° C. and 60° C. for reaction and activation for 4 hours to obtain completely cross-linked latex-antibody suspensions;
   (6) to (8) were the same with Example 2;
   (9) resuspended for later use.
3. Calibrator(s) and control material(s): same with Example 1.
4. Detection:

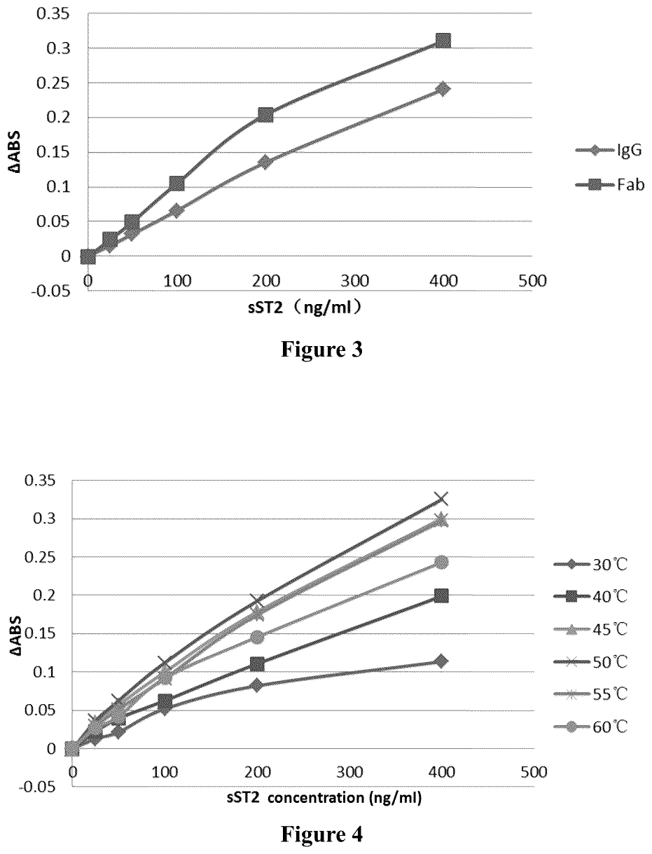

According to FIG. 3, it was shown that EDC at different temperatures had different activation effects on the latex functional groups (for example carboxyl), and finally had different effects on the absorbance of reagents. According to the results, preferably, boric acid buffer, 45° C. to 55° C. was the optimal activation environment.

### Example 4. Antibody Affinity of Fab Fragment Antibodies

1. The first reagent: same with Example 1.
2. The second reagent:
   methods of preparation:
   (1) same with Example 2;
   (2) each of 1 mg anti-sST2 monoclonal antibody (IgG type and Fab fragment) was added to 4.5 ml boric acid pH 9.0 buffer, respectively, to obtain antibody solutions;
   (3) same with Example 3;
   (4) same with Example 1;
   (5) the carbodiimide aqueous solution was added to the mixed solution of step (3) and placed at 50° C. for reaction and activation for 4 hours to obtain completely cross-linked latex-antibody suspensions;
   (6) to (8) were the same with Example 1;
   (9) resuspended for later use.
3. Calibrator(s) and control material(s): same with the examples.
4. Detection:

According to FIG. 4, it was shown that the Fab fragment antibodies had high antibody affinity and high detection absorbance. The detection sensitivity of reagents could be improved to a higher degree.

### Example 5. Optimization of Blocking of the Latex

1. The first reagent: same with Example 1.
2. The second reagent:
   methods of preparation:
   (1) same with Example 2;
   (2) each of 1 mg anti-sST2 Fab fragment recombinant monoclonal antibody was added to 4.5 ml boric acid pH 9.0 buffer, respectively, to obtain antibody solutions;
   (3) same with Example 3;
   (4) same with Example 1;
   (5) same with Example 4;
   (6) 2 ml of each was added to different blocking solutions for blocking overnight at room temperature. The composition of the blocking solution was shown in Table 1.

TABLE 1
The composition of the blocking solution

| | Concentration in the system |
|---|---|
| Components | (w/v) |
| Polyethylene glycol polyamine | 1% |
| BSA | 1% |
| Tween | 1% |
| Casein | 0.05% |
| Polyethylene glycol polyamine + BSA | 1% + 1% |

(7) to (8) were the same with Example 1;
   (9) resuspended for later use.
3. Calibrator(s) and control material(s): same with Example 1.
4. Detection:

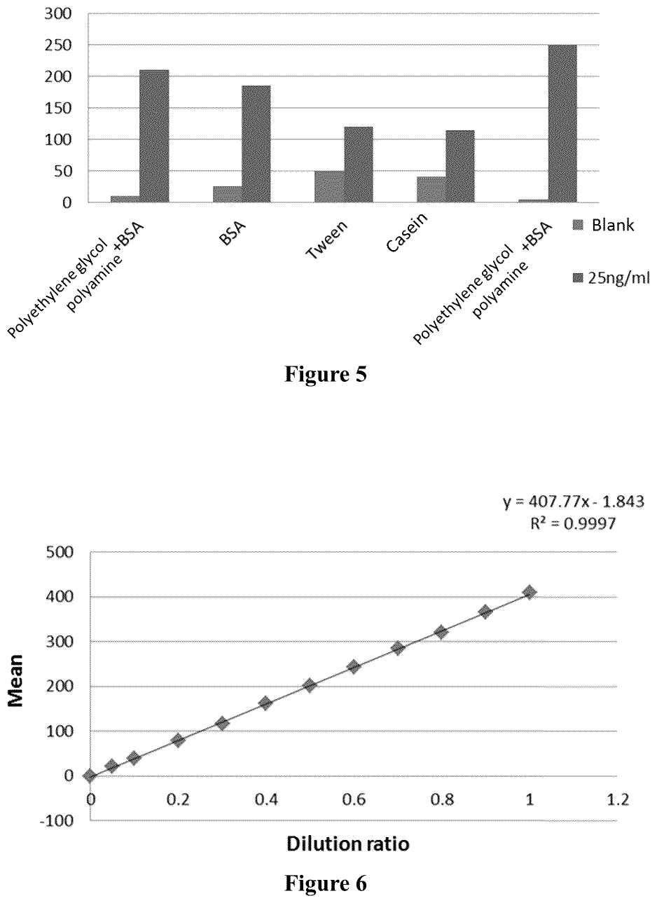

According to FIG. 5, it was shown that polyethylene glycol polyamine could effectively bind to the activated carboxyl that was not bound to antibodies, neutralize its charge and reduce the adsorption of the carboxyl to proteins upon activation. BSA could effectively attach to the surface of latex microspheres, reduce the adsorption of proteins onto the surface of latex microspheres.

### Example 6. Preparation of sST2 Detection Kit (Containing the Anti-Interference Composition)

1. The first reagent:
   150 mmol/L sodium chloride,
   1% surfactant (Brij 35),
   0.1% sodium azide,
   5 KU/L lipase,
   0.5% methacryloyloxy phosphatidylcholine,
   100 mmol/l Tris buffer, pH 7.4.
2. The second reagent comprises:
   0.15% w/v 400 nm latex microspheres,
   recombinant Fab fragment of monoclonal antibody 20 μg/ml,
   100 mmol/L glycine buffer pH 8.0,
   0.10% NaCl,
   0.5% methacryloyloxy phosphatidylcholine,
   0.10% NaN₃.

The preparation process of latex microspheres was the same with Example 5.
3. Calibrator(s) and control material(s): same with Example 1.
4. Detection of precision for the reagent: see Table 2.

TABLE 2
Precision of the reagents prepared in Example 6

| | Repeatability | Intermediate precision |
|---|---|---|
| | (n = 20) | (n = 80) |
| Quality control 1 (30 ng/ml) | 1.50% | 1.78% |
| Quality control 2 (100 ng/ml) | 0.90% | 1.01% |
| Sample (20 ng/ml) | 2.1% | 3.40% |

### Example 7. Preparation of sST2 Detection Kit (Containing the Blocker+the Anti-Interference Composition)

1. The first reagent:
   1% surfactant (Thesit),
   0.1% sodium azide,
   5 KU/L lipase,
   2% sodium cholate,
   5 mmol/l CaCl₂),
   2.0% methacryloyloxy phosphatidylcholine,
   3% blocker HBR-8^{TM},
   200 mmol/l Tris buffer, pH 7.4.
2. The second reagent comprises:
   same with Example 6.
3. Calibrator(s) and control material(s): same with Example 1.

### TEST EXAMPLES

### Test Example 1. Reagent Specificity

To adjust the first reagent, the introduction of surfactant and lipase system improved the detection accuracy of the reagent when detecting samples with high level of chyle.

The detection accuracy of the reagent for high RF samples was improved by adding a blocker. Detailed data can be seen in Table 3.

TABLE 3
Anti-interference effect

| | Example 6 | Example 7 |
|---|---|---|
| Interference | (ng/ml) | (ng/ml) |
| Control | 25.0 | 26.7 |
| 500 mg/dl hemoglobin | 24.8 | 25.2 |
| 1000 mg/dl triglyceride | 0.8 | 26.1 |
| 500 IU/L rheumatoid factors (RF) | 51.0 | 25.1 |
| 40 mg/dl bilirubin | 23.8 | 26.4 |

### Test Example 2. Linearity

High-concentration samples were used and diluted at a fixed ratio by using normal saline. The detection results were R²>0.99 (see FIG. 6).

### Test Example 3. Pre-Zone Effects

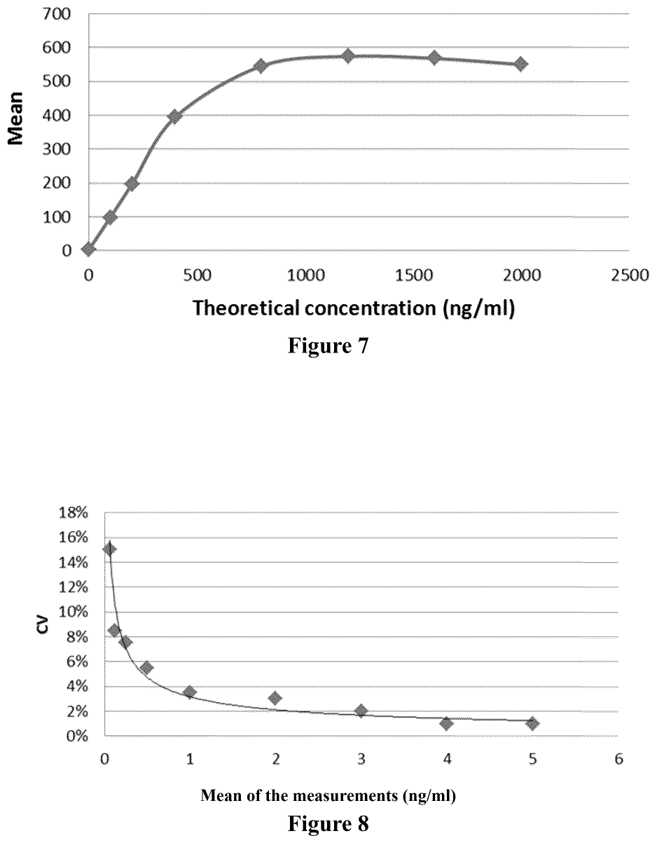

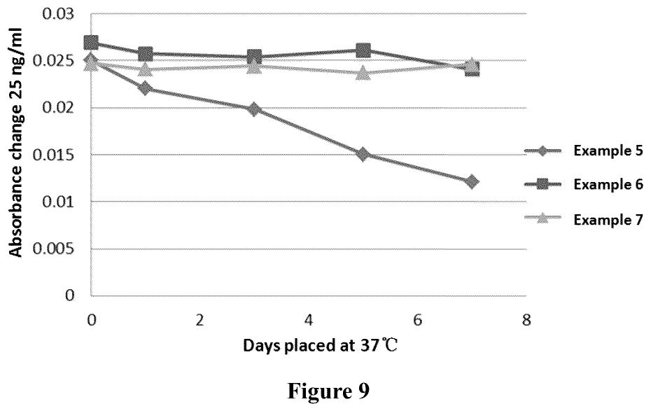

The antigen sST2 protein with theoretical concentration of 2000 ng/ml was used and diluted with normal saline at certain proportions. The samples with theoretical concentration of 2000 ng/ml were detected, and the detection result goes beyond the linear range (see FIG. 7).

### Test Example 4. Sensitivity

When using scatter immunoturbidity analysis, conventional samples and normal saline were diluted. Samples of each level were tested for 20 times and CV was calculated. CV<10% was deemed as LoQ.

The detection result of the kit of Example 7 was LoQ=0.1 ng/ml (see FIG. 8).

### Test Example 5. Establishment of Clinical Reference Range

Physical examination samples were collected, and grouped according to gender Heart disease, inflammatory disease, tumor and other related diseases were excluded. 120 cases of males and 120 cases of females were tested and analyzed, and the results were as follows:

TABLE 4
Clinical reference range

| | Male | Female |
|---|---|---|
| Mean value | 30.0 ng/ml | 20.5 ng/ml |
| Sd | 3.5 ng/ml | 3.7 ng/ml |
| 99% quantile | 42.1 ng/ml | 32.4 ng/ml |

### Test Example 6. Performance on Diagnosis of Disease

The kit of Example 7 was used with the clinical reference range: 0.1 ng/ml to 35 ng/ml for normal population, and the result determination was detected.

TABLE 5
Reference range

| | Mean value of sST2 | Positive |
|---|---|---|
| Diagnosis | (ng/ml) | results |
| Healthy controls (n = 50) | 30 ng/ml | 3 cases (94%) |
| Patients (n = 50) | 156 ng/ml | 35 cases (70%) |

### Test Example 7. Stability of the Reagents

The stability test was carried out using the kits in Examples 5 to 7. The reagents were added for the stability test at 37° C. The kits of Examples 6 and 7 exhibited the best stability. After accelerating at 37° C. for 7 days, the calibrated absorbance of the reagents showed no significant changes.

1-9. (canceled)

10. A method for fixing polypeptides to latex microspheres, comprising:
providing a polypeptide in a buffer;
providing latex microspheres in the buffer;
mixing the polypeptide and the latex microspheres at a temperature of 10° C. to 35° C. for 6 to 10 hours to obtain a first mixture by shaking and mixing;
providing a crosslinking agent;
contacting the crosslinking agent with the first mixture at a temperature of 30° C. to 60° C. for 2 to 5 hours to obtain latex microspheres cross-linked with the polypeptide;
wherein the polypeptide carries at least one functional group selected from the group consisting of amino, carboxyl, hydroxyl, and sulfhydryl;
wherein the polypeptide is selected from the group consisting of an antibody, an antigen-binding fragment, an antigen, an enzyme, and a recombinant protein;
wherein a surface functional group of the latex microsphere is selected from the group consisting of carboxyl, sulfhydryl, chloromethyl, and no surface functional group;
wherein the latex microspheres have an average particle size of 350 nm to 450 nm;
wherein the buffer is selected from the group consisting of HEPES buffer, glycine buffer, Tris buffer, PBS buffer, MOPS buffer, and boric acid buffer;
wherein the buffer provides a pH of 7.0 to 9.0; and
wherein a concentration of the buffer is 10 mmol/L to 1000 mmol/L.

11. The method of claim 10, wherein:
the mixing is performed at a temperature of 20° C. to 25° C. for 7 to 9 hours,
the crosslinking agent comprises carbodiimide;
the contacting is performed at a temperature of 45° C. to 55° C. for 2 to 4 hours;
the buffer comprises boric acid buffer at pH 9.0; and
the concentration of the buffer is 10 mmol/L to 1000 mmol/L.

12. The method of claim 10, further comprising blocking the latex microspheres cross-linked with the polypeptide by:
contacting the latex microspheres cross-linked with the polypeptide with a blocking system at a temperature of 15° C. to 28° C. for 1 hour to 24 hours;
wherein the blocking system comprises a blocking agent, a buffer having a pH of 5.0 to 8.0, and a surfactant;
wherein the blocking agent is selected from the group consisting of: 0.1% to 5.0% w/v polyethylene glycol having a molecular weight of 1000 to 5000, 0.1% to 5.0% w/v polyethylene glycol polyamine having a molecular weight of 1000 to 5000, 2.0% to 10.0% w/v bovine serum albumin, 2.0% to 10.0% w/v dextran gel, and 2.0% to 10.0% w/v casein;
wherein the buffer of the blocking system is selected from the group consisting of: phosphate buffer, glycine buffer, and HEPES buffer; and
wherein the surfactant is selected from the group consisting of: 2.0% to 8.0% w/v Triton X, 2.0% to 8.0% w/v Tween, and 2.0% to 8.0% w/v AEO.

13. The method of claim 12, further comprising one or more steps selected from the group consisting of:
rinsing the latex microspheres cross-linked with the polypeptide;
centrifuging and collecting the latex microspheres cross-linked with the polypeptide; and
packaging the latex microspheres cross-linked with the polypeptide.

14. A method of performing immunoturbidimetric detection, comprising:
providing an immunoturbidimetric detection kit comprising an anti-interference composition; and
performing immunoturbidimetric detection using the kit;
wherein the anti-interference composition comprises 0.1% to 10% w/v surfactant and 0.5 KU/L to 10 KU/L lipid digestive enzyme;
wherein the surfactant is selected from the group consisting of: Triton X, Tween, AEO, Thesit, Brij, and NP; and
wherein the lipid digestive enzyme is selected from the group consisting of: lipase and triglyceride oxidase.

15. The method of claim 14, wherein the immunoturbidimetric detection kit is selected from the group consisting of:
(a) a first kit comprising:
a first reagent comprising: 200 mmol/L sodium chloride, 1% w/v surfactant, 0.1% w/v sodium azide, and 100 mmol/L HEPES buffer, pH 8.0;
a second reagent comprising: 0.1% w/v 400 nm latex microspheres and 30 g/mL anti-sST2 monoclonal antibody;
(b) a second kit comprising:
a first reagent comprising: 150 mmol/L sodium chloride, 1% w/v surfactant, 0.1% w/v sodium azide, 5 KU/L lipase, 0.5% methacryloyloxy phosphatidylcholine, and 100 mmol/L Tris buffer, pH 7.4;
a second reagent comprising: 0.15% w/v 400 nm latex microspheres, 20 g/mL sST2 antibody Fab fragment, 100 mmol/L glycine buffer at pH 8.0, 0.1% w/v NaCl, 0.5% w/v methacryloyloxy phosphatidylcholine, and 0.1% w/v NaN₃; and
(c) a third kit comprising:
a first reagent comprising: 1% w/v surfactant, 0.1% w/v sodium azide, 5 KU/L lipase, 2% w/v sodium cholate, 5 mmol/L CaCl₂, 2.0% w/v methacryloyloxy phosphatidylcholine, 3% w/v blocker HBR-8^{TM}, and 200 mmol/L Tris buffer, pH 7.4; and
a second reagent comprising: 0.15% w/v 400 nm latex microspheres, 20 g/mL sST2 antibody Fab fragment, 100 mmol/L glycine buffer at pH 8.0, 0.1% w/v NaCl, 0.5% w/v methacryloyloxy phosphatidylcholine, and 0.1% w/v NaN₃;
wherein the surfactant is selected from the group consisting of: Triton X, Tween, AEO, Thesit, Brij, and NP.

16. The method of claim 15, wherein the kit further comprises at least one of a calibrator and a control material.

17. The method of claim 16, wherein:
the calibrator comprises sST2 protein at a concentration selected from the group consisting of: 25 ng/mL, 50 ng/mL, 100 ng/mL, 200 ng/mL, and 400 ng/mL;
the control material comprises sST2 protein at a concentration selected from the group consisting of: 30 ng/mL and 100 ng/mL;
the calibrator or the control material comprises a buffer selected from the group consisting of: phosphate buffer, HEPES buffer, MOPS buffer, MES buffer, and PIPES buffer;
the buffer has a concentration of 5 mmol/L to 250 mmol/L;
the buffer has a pH of 4.0 to 8.0;
the calibrator or the control material comprises 1.0% to 10% w/v protectant selected from the group consisting of: bovine serum albumin, saccharide, and alcohol; and
the calibrator or the control material comprises 0.05% to 1.5% w/v preservative selected from the group consisting of: sodium azide, PC, and dithiothreitol.

18. The method of claim 17, wherein the calibrator or the control material comprises sST2, 100 mmol/L phosphate buffer at pH 5.5, 150 mmol/L NaCl, 5% w/v BSA, 10% w/v sorbitol, and 0.1% w/v NaN₃.
